(19) United States
(12) Patent Application Publication
(10) Pub. No.: US 2008/0200507 A1
(43) Pub. Date: Aug. 21, 2008

(54) Crystalline Non-Solvated Methanesulfonic Acid Salt of 1-(4-(2-Piperidinylethoxy)Phenoxy)-2-(3-Hydroxyphenyl)-6-Hydroxynaphthalene

(76) Inventor: Jeanette Tower Dunlap, Indianapolis, IN (US)

Correspondence Address: ELI LILLY & COMPANY, PATENT DIVISION, P.O. BOX 6288, INDIANAPOLIS, IN, 46206-6288, US

(21) Appl. No.: 11/995,852

(22) PCT Filed: Jul. 27, 2006
(86) PCT No.: PCT/US2006/028956

§ 371 Date: Jan. 16, 2008

Related U.S. Application Data
(60) Provisional application No. 60/703,851, filed on Jul. 29, 2005.

(51) Int. Cl. A61K 31/4453; C07D 211/22; A61P 35/00

(52) U.S. Cl. 514/319; 546/206

(57) ABSTRACT

The present invention relates to the mesylate salt of 1-(4-(2-piperidinylethoxy)phenoxy)-2-(3-hydroxyphenyl)-6-hydroxynaphthalene.

### BACKGROUND OF THE INVENTION

Uterine leiomyoma/leiomyomata (uterine fibroid disease) is a clinical problem that goes under a variety of names, including uterine fibrosis, uterine hypertrophy, uterine leiomyomata, myometrial hypertrophy, fibrosis uteri, and fibrotic metritis. Essentially, uterine fibrosis is a condition where there is an inappropriate deposition of fibroid tissue on the wall of the uterus. This condition is a cause of dysmenorrhea and infertility in women.

Endometriosis is a condition of severe dysmenorrhea, which is accompanied by severe pain, bleeding into the endometrial masses or peritoneal cavity and often leads to infertility. The symptoms' cause appears to be ectopic endometrial growths that respond inappropriately to normal hormonal control and are located in inappropriate tissues. Because of the inappropriate locations for endometrial growth, the tissue seems to initiate local inflammatory-like responses causing macrophage infiltration and a cascade of events leading to initiation of the painful response. Evidence suggests that a cause of uterine fibrosis and endometriosis is an inappropriate response of fibroid tissue and/or endometrial tissue to estrogen.

Many publications have appeared within the last ten years disclosing selective estrogen receptor modulators (SERMs), e.g., WO 98/08797. The clinical use of SERM compounds for the treatment of uterine fibroid disease and/or endometriosis, particularly in pre-menopausal women, has been hampered, however, by the potential of said compounds to have significant ovarian stimulatory effects at the doses necessary to see efficacy for fibroid or endometriosis treatment.

A particular SERM compound of interest disclosed in WO 98/08797 is the hydrochloride salt of 1-(4-(2-piperidinylethoxy)phenoxy)-2-(3-hydroxyphenyl)-6-hydroxynaphthalene. This compound is disclosed therein as “Example 3”. Example 3 described preparing “amorphous” hydrochloride from a “residue” that contained 1-(4-(2-piperidinylethoxy)phenoxy)-2-(3-hydroxyphenyl)-6-hydroxynaphthalene (hereafter referred to as “GS II”). Although the GS-II, and the hydrochloride salt thereof, prepared by the procedures taught in WO 98/08797 could be used as pharmaceuticals, it would be highly desired and advantageous to find a crystalline salt form of GS-II that did not contain water nor an organic solvent within its crystal lattice, that is non-hygroscopic, that is water soluble and which could be efficiently prepared and formulated on a commercial scale.

### SUMMARY OF INVENTION

The present invention relates to the mesylate salt of 1-(4-(2-piperidinylethoxy)phenoxy)-2-(3-hydroxyphenyl)-6-hydroxynaphthalene, that is, the mesylate salt of a compound of the formula:

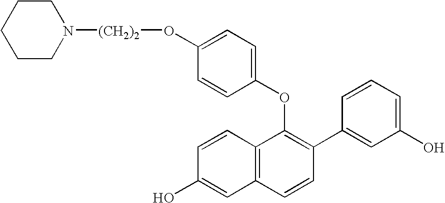

hereafter referred to as “GS II mesylate”.

The present invention further relates to a crystalline non-solvated form of GS II mesylate characterized by an X-ray diffraction pattern which comprises the following peaks: 13.0±0.1, 13.6±0.1, 18.6±0.1, 19.0±0.1, 21.0±0.1 and 22.3±0.1° in 2θ; when the pattern is obtained from a copper radiation source (CuKα, λ=1.54056 Å). This same crystalline form may also be identified by peaks at 6.4±0.1, 7.9±0.1 and 9.3±0.1° in 2θ.

The present invention also relates to a pharmaceutical composition containing a salt of the present invention, and a pharmaceutical carrier. In another embodiment, the pharmaceutical compositions of the present invention may be adapted for use in treating endometriosis and/or uterine leiomyoma.

The present invention also relates to methods for preventing and treating endometriosis and/or uterine leiomyoma which comprise administering to a patient in need thereof an effective amount of a salt of the present invention.

In addition, the present invention relates to a salt of the present invention for use in treating endometriosis and/or uterine leiomyoma. The present invention is further related to the use of a salt of the present invention for the manufacture of a medicament for treating endometriosis and/or uterine leiomyoma.

### BRIEF DESCRIPTION OF THE FIGURE

FIG. 1 is a representative XRD pattern for crystalline non-solvated GS II mesylate.

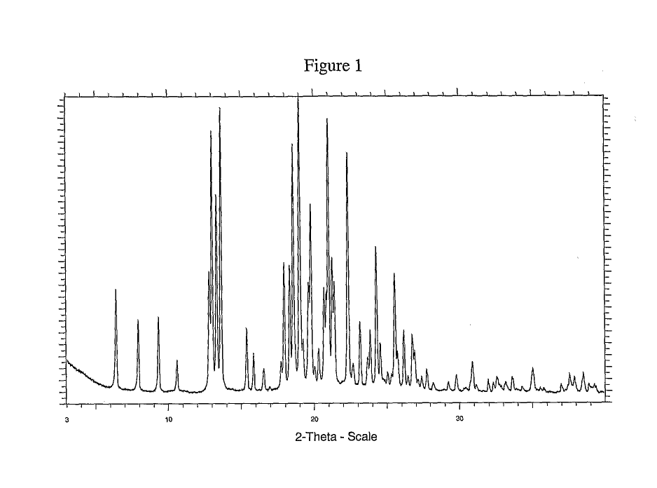

### DETAILED DESCRIPTION OF THE INVENTION

Applicants have found that the hydrochloride salt of GS II can be prepared in at least two crystalline hydrated forms (F-I and F-II). F-I is a hemi-hydrate that can convert to the sesqui-hydrated, hygroscopic F-II. Although these forms of GS II hydrochloride may be useful as pharmaceuticals, F-I's lack of stability to aqueous environments (humidity) and F-II's hygroscopicity hamper their use in large-scale production and formulation of these two active ingredients.

X-ray diffraction (XRD) analysis and automated in situ salt screening of GS II revealed that GS II free base and the citrate and maleate salts thereof did not form crystalline solids. In addition, although a crystalline form of GS II lactate was found in the in situ screen, said salt was solvated (as measured by differential thermal/thermogravimetric analyses, the lactate had a weight percentage loss of 3% from ambient to 175° C.).

Poorly crystalline and/or amorphous materials are typically less desirable than highly crystalline materials for formulation processing. Amorphous compounds are chemically and physically less stable as they tend to adsorb significant amounts of water. The adsorption of water by an amorphous material in a gelatin capsule, for example, may cause the capsule to shrink or buckle as moisture is transferred from the capsule to the amorphous component. In addition, amorphous compounds have a tendency to precipitate out of solutions containing them. If an amorphous drug substance precipitates from a delivery solution, the dissolution and bioavailability properties of the drug may be negatively affected.

In addition, it is generally not desirable to formulate pharmaceuticals containing substantial amounts of organic solvent due to potential solvent toxicity to the recipient thereof and changes in potency of the pharmaceutical as a function of the solvent. In addition, from a manufacturing perspective, it is also generally less desirable to prepare non-crystalline materials whenever said preparation involves a collection of the final product via filtration. Such filtrations are often more difficult to perform when the material collected is non-crystalline. Moreover, it is also generally less desirable, from a manufacturing perspective, to formulate pharmaceuticals containing substantial amounts of water (hydrates) because the level of hydration will typically be some function of the relative humidity at which the pharmaceutical is produced and stored. In other words, potency variability is typically more problematic with a hydrate relative to its anhydrous form.

The in situ salt screening described above also revealed that the crystalline fumarate, succinate, sulfate, and tosylate salts of GS II had relatively low in situ aqueous solubility. When delivering a drug via the oral route, it is generally preferred to find a form of that drug that is soluble in water. In general, as aqueous solubility increases, the potential for absorption of the drug in the gut (and ultimate bioavailability) increases as well. Higher bioavailability can result in lower variability in clinical exposure and thus give the physician an advantage in correctly dosing-the patient within the therapeutic window. Using the aqueous in situ solubility as a guide, and the robustness of the same crystalline form isolated via automation, the crystalline mesylate, lactate, tartrate and phosphate salts of GS II were identified as candidates for further evaluation. Separately, the crystalline acetate salt of GS II was identified and also further characterized.

Four of these salts (the crystalline acetate, mesylate, lactate and phosphate salts of GS II) were administered to monkeys and blood levels of GS II and its conjugates were measured. The salt form that gave the largest in vivo exposure of GS II (ng hr/ml of GS II and of its conjugates) in this study was the crystalline mesylate salt form (non-solvated). Some additional physical properties of crystalline non-solvated GS II mesylate are disclosed below in Table 1.

TABLE 1

| Physical Property | Mesylate |
|---|---|
| % Volatiles (Thermo Gravimetric Analysis) | <1% (25 to 216° C.) |
| % Moisture Adsorbed @80% RH | <1 |

In summary, GS II mesylate may be prepared in a non-solvated crystalline form that is non-hygroscopic and that can provide significant in vivo exposure of GS II in monkeys.

### Characterization of Crystalline Non-Solvated GS II Mesylate

Being a non-solvated crystal form, it should be understood that crystalline non-solvated GS II mesylate is an anhydrous salt form.

The XRD pattern for crystalline non-solvated GS II mesylate features sharp peaks and a flat baseline, indicative of a highly crystalline material. The angular peak positions in 2θ and corresponding I/I_{O} data for all peaks with intensities equal to or greater than 10% of the largest peak for crystalline non-solvated GS II mesylate are shown in Table 2. All data in Table 2 is expressed with an accuracy of ±0.1° in 2θ.

TABLE 2

| Angle | |
|---|---|
| (degrees 2θ) | I/Iₒ (%) |
| 6.4 | 34.1 |
| 7.9 | 24.4 |
| 9.3 | 25.6 |
| 10.8 | 10.9 |
| 12.8 | 41.3 |
| 13.0 | 89.8 |
| 13.3 | 67.6 |
| 13.6 | 97.6 |
| 15.4 | 21.4 |
| 15.8 | 12.6 |
| 17.9 | 43.4 |
| 18.3 | 42.2 |
| 18.6 | 83.9 |
| 19.0 | 100.0 |
| 19.2 | 16.4 |
| 19.6 | 35.8 |
| 19.8 | 62.8 |
| 20.3 | 12.6 |
| 20.7 | 33.6 |
| 20.9 | 32.4 |
| 21.0 | 91.8 |
| 21.3 | 43.9 |
| 21.4 | 35.6 |
| 22.3 | 80.0 |
| 23.2 | 21.6 |
| 23.9 | 18.8 |
| 24.3 | 47.7 |
| 24.6 | 14.4 |
| 25.6 | 38.7 |
| 25.7 | 12.2 |
| 26.2 | 19.6 |
| 26.8 | 17.8 |
| 26.9 | 13.0 |
| 30.9 | 10.1 |

It is well known in the crystallography art that, for any given crystal form, the relative intensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g., The United States Pharmacopeia #23, National Formulary #18, pages 1843-1844, 1995. Furthermore, it is also well known in the crystallography art that, for any given crystal form, the angular peak positions may vary slightly. For example, peak positions can shift due to a variation in the temperature at which a sample is analyzed, sample displacement, or the presence or absence of an internal standard. In the present case, a peak position variability of ±0.1° in 2θ will take into account these potential variations without hindering the unequivocal identification of crystalline non-solvated GS II mesylate.

Based on peak intensities as well as peak position, crystalline non-solvated GS-II mesylate may be identified by the presence of peaks at 13.0±0.1, 13.6±0.1, 18.6±0.1, 19.0±0.1, 21.0±0.1 and 22.3±0.1° in 2θ; when the pattern is obtained from a copper radiation source (λ=1.54056 Å). The presence of crystalline non-solvated GS-II may also be identified by peaks at 6.4±0.1, 7.9±0.1 and 9.3±0.1° in 2θ; when the pattern is obtained from a copper radiation source (λ=1.54056 Å).

Crystalline non-solvated GS-II mesylate may also be characterized by solid-state NMR spectroscopy. Solid-state ¹³C chemical shifts reflect the molecular structure and electronic environment of the molecule in the crystal. The spectrum for crystalline non-solvated GS-II mesylate comprises isotropic peaks at the following chemical shifts: 41.9, 111.0, 114.6, 115.2, 116.0, 117.3, 119.1, 119.9, 121.1, 122.4, 125.7, 127.9, 128.5, 129.9, 137.7, 140.4, 146.8, 153.0 and 157.4 ppm.

### Characterization Methods

The XRD pattern is obtained from 3 to 400 in 2θ using a Bruker D4 Endeaver X-ray powder diffractometer, equipped with CuKα source (λ=1.54056 Å) and a Vantec detector.

¹³C Cross polarization/magic angle spinning (CP/MAS) NMR (solid-state NMR or SSNMR) spectrum is obtained using a Varian Unity Inova 400 MHz NMR spectrometer operating at a carbon frequency of 100.578 MHz and equipped with a complete solids accessory and a Chemagnetics 4.0 mm T3 probe. Acquisition parameters are as follows: 90° proton r.f. pulse width 4.0 μs, contact time 2.0 ms, pulse repetition time 20 s, MAS frequency 10 kHz, spectral width 50 kHz, and acquisition time 50 ms. Chemical shifts are referenced to the methyl group of hexamethylbenzene (δ=17.3 ppm) by sample replacement.

### Synthesis

### Preparation 1

### 1-(4-(2-Piperidinylethoxy)phenoxy)-2-(3-methoxyphenyl)-6-methoxynaphthalene, hydrochloride

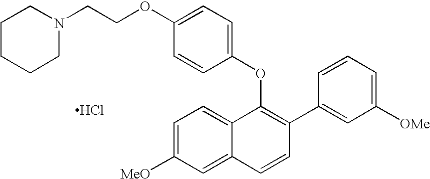

To a 12-L four-neck round-bottom flask equipped with a mechanical stirrer, thermocouple, reflux condenser and a three-way valve connected to a nitrogen source and house vacuum, charge a solution of 6-methoxy-α-tetralone (750 g, 4.26 moles) in tetrahydrofuran (THF; 3750 ml) at ambient temperature. Apply house vacuum until a gentle reflux is observed degassing the solution. Purge the round-bottom with nitrogen via the three-way valve. Repeat this procedure two additional times. Solid bis palladium (0) tris (dibenzylideneacetone) (Pd₂(dba)₃; 19.5 g, 0.0213 moles, 0.005 eq.) and bis[2-(diphenylphosphino)phenyl]ether (DPE-Phos; 23.0 g, 0.0426 moles, 0.01 eq.) is charged and degas the resulting solution as before. Charge solid sodium t-butoxide (421 g, 4.38 moles, 1.03 eq.) and follow immediately by neat 3-bromoanisole (820 g, 555 ml, 4.38 moles, 1.03 eq.). Degas the reaction mixture for a third time, then stir vigorously under positive nitrogen pressure. Allow the reaction to cool to 35° C. then use a heating mantle to maintain a temperature of 32° C. for four days. Remove the heating mantle and quench the reaction mixture slowly by addition of water (2 L) at such a rate to keep the reaction temperature below 36° C. Transfer the vessel contents to a 22-L bottom-outlet flask equipped with a mechanical stirrer. Charge ethyl Acetate (4 L) and water (4 L) and stir the contents. Isolate the aqueous layer and extract with ethyl acetate (2 L). Combine the organic layers and wash with water (4 L) followed by saturated aqueous sodium chloride (4 L). Dry the organic layer with granular sodium sulfate and filter the mixture directly over a 440-g pad of 100-200 mesh Florisil in a sintered glass funnel (approximately 2 inches deep). Wash the pad with ethyl acetate (2 L) and concentrate the filtrate in vacuo.

Dissolve the oil in tert-butyl methyl ether (4 L) and filter slowly over a 500-g pad of 100-200 mesh Florisil packed in a sintered funnel (approximately 2 inches). Transfer the filtrate to a 12-L 4-neck flask equipped with a mechanical stirrer and a positive nitrogen inlet. Stir the solution slowly at ambient temperature for 16 hours to form a crystalline mixture. Filter the solid and rinse with tert-butyl methyl ether (500 mL). Dry the material in vacuo at 40° C. to yield 844 g (70%) of 6-methoxy-2-(3-methoxyphenyl)-3,4-dihydro-2H-naphthalen-1-one. ¹H NMR (DMSO-d₆, 300 MHz): δ2.17-2.39 (m, 2H), 2.88-2.96 (m, 1H), 3.01-3.12 (m,1H), 3.70 (s, 3H), 3.78-3.84 (m, 1H), 3.82 (s, 3H), 6.70-6.78 (m, 2H), 6.79-6.82 (m, 1H), 6.89-6.92 (m, 2H), 7.17-7.23 (m, 1H), 7.84-7.87 (dd, 1H)

Charge to a 12-L 4-neck round-bottom flask equipped with a mechanical stirrer, reflux condenser, heating mantle, thermocouple and nitrogen inlet 6-methoxy-2-(3-methoxyphenyl)-3,4-dihydro-2H-naphthalen-1-one (760 g, 2.69 moles), Hyflo® (190 g) and toluene (3800 ml) and stir the resulting suspension vigorously under a positive nitrogen blanket via a bubbler. Add to the mixture PBr₃ (801 g, 280 ml, 2.96 moles, 1.1 eq.) quickly via graduated cylinder. Heat the reaction mixture to reflux and stir overnight. After 18 hours, remove the heating mantle and cool the reaction mixture to 35° C. Filter the slurry over a 1-inch deep pad of Hyflo® in a 3-L sintered funnel. Slowly add the orange filtrate to a solution of NaCO₃ (1.5 kg) in water (8 L). Stir the biphasic mixture vigorously for 40 minutes, and separate the organic layer. Wash the organic layer two times with a solution of Na₂CO₃ (500 g) in water (4 L). Dry the organic layer over granular sodium sulfate, filter and concentrate in vacuo to yield 804 g of crude 4-bromo-7-methoxy-3-(3-methoxyphenyl)-1,2-dihydronaphthalene.

Slurry 1591 g of 4-bromo-7-methoxy-3-(3-methoxyphenyl)-1,2-dihydronaphthalene in tert-butyl methyl ether (3182 mL). Heat the slurry to 35° C. and stir for 2 hours, then cool to ambient temperature overnight with stirring. Filter the slurry and dry the product further in vacuo at 40° C. for 48 hours to afford 1.073 kg (58.4%) of 4-bromo-7-methoxy-3-(3-methoxyphenyl)-1,2-dihydronaphthalene as a solid. ¹H NMR (DMSO-d₆, 300 MHz): δ2.64-2.70 (m, 2H), 2.90-2.94 (m, 2H), 3.8 (s, 3H), 3.81 (s, 3H), 6.85-6.9 (m, 3H), 6.92-6.97 (m, 2H), 7.31-7.36 (m, 1H), 7.53-7.56 (dd, 1H).

Charge 4-bromo-7-methoxy-3-(3-methoxyphenyl)-1,2-dihydronaphthalene (536.66 g, 1.55moles) followed by THF (1610 mL) to a 12-L 4-neck round-bottom flask equipped with a mechanical stirrer, reflux condenser, thermocouple, heating mantle and positive nitrogen pressure inlet and stir the contents at ambient temperature. Charge dichlorodicyanoquinone (DDQ; 366 g, 1.61 moles, 1.04 eq.) to the solution and heat the reaction mixture to 40° C. Stir the reaction overnight at 40° C. Charge additional DDQ (4 g) and stir the reaction for 3 hours. Add additional DDQ (10 g) and stir the reaction vigorously for three days. Add a 0.5 M solution of sodium hydroxide (5365 ml, 2.7 moles) and stir the reaction mixture overnight at 40° C. Cool the mixture to ambient temperature and add ethyl acetate (8 L). Separate the organic layer and wash twice with a solution of 0.5 N NaOH (4 L) followed by washes with water (4 L) then saturated aqueous sodium chloride (4 L). Dry the organic layer over granular Na₂SO₄ and concentrate to yield a semisolid. Chromatograph the semisolid on 3 kg of silica gel eluting with dichloromethane. Collect fractions and pool to afford an off-white solid. Slurry the solid in tert-butyl methyl ether (1.5 L). Isolate the solid product via filtration and wash with tert-butyl methyl ether (150 mL). Dry the filter cake in vacuo overnight at 40° C. to afford 1-bromo-6-methoxy-2-(3-methoxyphenyl)naphthalene: 500 g, 94%. ¹H NMR (DMSO-d₆, 300 MHz): δ3.78 (s, 3H), 3.91 (s, 3H), 6.96-7.00 (m, 3H), 7.33-7.46 (m, 4H), 7.88-7.91 (d, 1H), 8.16-8.20 (d, 1H)

Add toluene (3.00 L) and 1-bromo-6-methoxy-2-(3-methoxyphenyl)naphthalene (2.61 kg, 1.46 mol) and 4-(2-piperidin-1-yl-ethoxy)phenol (341.09 g, 1.54 mmol, 1.06 equiv) to a 12-L 4-neck round-bottom flask fitted with Dean-Stark trap and nitrogen vent and initiate overhead stirring. Add cesium carbonate (570.32 g, 1.75 mol) and cuprous chloride (7.27 g, 73.44 mmol), and stir and heat the resulting mixture stirred to reflux, with collection of water in the Dean-Stark trap, for 48 hours. Cool the reaction to room temperature over 1 hour, and transfer to a 22-L bottom outlet flask. Add 1 N sodium hydroxide (9 L) and stir the mixture for 15 minutes, then allow the layers to separate for 1 hour. Remove the bottom aqueous layer, along with tarry interfacial material. Perform a second wash with NaOH (9 L) in similar fashion, followed by a wash with 10% aqueous ammonia (9 L). Break up the emulsion, if formed, by mild agitation. Separate the layers, filter the organic layer through Hyflo® and rinse with toluene (1 L). Transfer the combined organic solution to a 12-L 3-neck vessel and heat to reflux, collecting distillate until approximately 4 L of solution remained. Cool the solution and maintained at 50° C. and add ethyl acetate (4.5 L), followed by ethanol (2B-3, 164 mL, 130 g, 2.82 moles). Add to this stirring solution acetyl chloride (0.21 L, 0.23 kg, 2.88 mol) over 1 hour. Cool the mixture to 20-25° C. over 1 hour, and stir overnight at room temperature. Filter the resulting slurry and rinse the filter cake with a 1:1 mixture of toluene:ethyl acetate (4 L) and dry in vacuo for 40 hours to afford a tan solid: 923.0 g (1.77 mol, 67.7%). Add this solid to a 12-L round-bottom flask and add acetonitrile (4.5 L). Heat the resulting slurry to reflux for 30 minutes, then cool to 5° C. and stir for 2 hours. Filter and dry further in vacuo for 3 days to afford the title compound as a tan solid: 781.9 g (1.50 mol, 57.4%).

### EXAMPLE 1

### Crystalline Non-Solvated 1-(4-(2-piperidinylethoxy)phenoxy)-2-(3-hydroxyphenyl)-6-hydroxynaphthalene, methanesulfonate

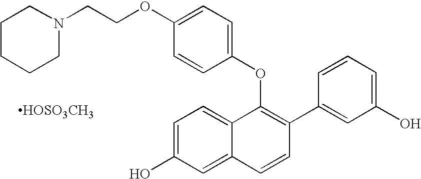

Add 1-(4-(2-piperidinylethoxy)phenoxy)-2-(3-methoxyphenyl)-6-methoxynaphthalene, hydrochloride (501.3 g, 963.9 mmol) and dichloromethane (3.5 L) to a 12-L 3-neck round-bottom flask. Heat the resulting solution to reflux under dry nitrogen, and collect 1000 mL of dichloromethane. Drain the distillate and cool the solution to below 5° C. under positive pressure of nitrogen. Add boron trichloride (677.3 g, 5.768 mol, 6 equiv) subsurface to the solution below 6° C. Allow the resulting solution to warm to room temperature and stir for 25 hours, then cooled to below 5° C., and add dropwise over 1 hour to precooled (1.7° C.) degassed methanol under nitrogen (3.5 L), maintaining temperature below 10° C. Distill the resulting solution under vacuum at 35° C. until 2.5 L had been removed. Add additional degassed methanol (5 L), and continue distillation under vacuum until 5 L distillate has been collected. Add the resulting methanol solution via FMI pump to a stirring mixture of methyl isobutyl ketone (4 L) and a solution of saturated aqueous NaHCO₃ (13 L). Following completion of addition, separate the layers and hold the organic layer overnight in refrigeration. Heat the organic layer to 50° C. and add methanesulfonic acid (64 mL, 963.91 mmol, 1.0 equiv) over 30 minutes, followed by seeding with title compound and stirring for an additional 30 minutes. Vacuum distill the resulting slurry at 50° C., collecting 2200 mL of distillate. Add methyl isobutyl ketone (2.5 L), followed by a second vacuum distillation at 50° C., collecting an additional 2100 mL of distillate. Add additional methyl isobutyl ketone (1.5 mL), and allow the resulting slurry to cool to room temperature over 1.5 hours. Cool the slurry to 0° C. and hold at that temperature for 1 hour, then filter. Rinse the filter cake with methyl isobutyl ketone (1 L) and dry further by pulling air through the cake. Further dry the solid in vacuo at 65° C. for 12 hours to afford 1-(4-(2-piperidinylethoxy)phenoxy)-2-(3-hydroxyphenyl)-6-hydroxynaphthalene, methanesulfonate as a tan solid: 520.0 g (942.61 mmol, 97.8%). Mp: 223.0-224.5° C.; Potency (avg. of 2 runs): 95.81%; Potency corrected yield: 498.21 g, 93.7%.

Add 1-(4-(2-piperidinylethoxy)phenoxy)-2-(3-hydroxyphenyl)-6-hydroxynaphthalene, methanesulfonate (488.90 g, Potency 95.81%) from the experiment above to a 5-L 4-neck round-bottom flask fitted with condenser, overhead stirring paddle and internal thermocouple. Add methanol (1.5 L) and stir the resulting slurry. Add methyl isobutyl ketone (1.5 L), and heat the resulting slurry to reflux for 16 hours using a timer. Cool the slurry overnight to room temperature, then cool to 0° C. and hold at that temperature for 1 hour. Filter then rinse the filter cake rinsed with room temperature methyl isobutyl ketone (1 L) and dry by pulling air through the cake. Further dry the solids in vacuo at 60° C. overnight to afford the title compound as an off-white solid: 402.4 g (906.35 mmol, 82.3%). Mp: 225.9-226.9° C.; Potency (avg. of 2 runs): 99.18%; Potency corrected yield: 399.10 g, 81.6%.

### Formulation (Pharmaceutical Composition)

A salt of the present invention, in particular the crystalline non-solvated GS II mesylate, is preferably formulated in a dosage unit form, i.e., in an individual delivery vehicle, for example, a tablet or capsule, prior to administration, preferably oral administration, to the recipient patient. The term “patient” means female humans (women) and non-human female animals such as companion animals (dogs, cats, horses and the like). The preferred patient is a woman. A particularly preferred patient in the context of uterine fibroid and/or endometriosis treatment is a premenopausal woman. A particularly preferred patient in the context of osteoporosis is a postmenopausal woman.

The present pharmaceutical compositions are prepared by known procedures using well-known and readily available ingredients. The term “pharmaceutical” when used herein as an adjective means substantially non-deleterious. In making the compositions of the present invention, a salt of the present invention (preferably crystalline non-solvated GS II mesylate) will usually be mixed with a carrier, or diluted by a carrier, or enclosed within a carrier. When the carrier serves as a diluent, it may be a solid, semisolid or liquid material that acts as a vehicle, excipient or medium for the active ingredient. The compositions are preferably in a form suitable for oral delivery such as a tablet or capsule.

### Formulation Examples

Formulation 1

| Ingredient | Mg per capsule |
|---|---|
| Crystalline Non-Solvated GS II Mesylate | 122.1 |
| Mannitol | 141.3 |
| Microcrystalline Cellulose (MCC) | 52.5 |
| Hydroxypropylmethyl cellulose (HPMC) | 14.0 |
| Sodium lauryl sulfate (SLS) | 7.0 |
| Sodium starch glycolate | 10.5 |
| Magnesium Stearate | 2.6 |
| TOTAL | 350 |

Make a 10% by weight SLS aqueous spray solution. Screen the mannitol, the MCC, the crystalline non-solvated GS II mesylate and HPMC through a security screen into a granulator bowl. Blend the contents of the granulator bowl. Add the spray solution to the blended powders while mixing. After complete addition of the spray-solution, switch to water and continue spraying until granulation process complete. Spread the granulated materials on paper lined trays for drying or dry the granulated materials in a fluid bed dryer. Remove the dried granulation and mill. Place the milled material into a suitable blender. Pass the sodium starch glycolate through a security screen and add this ingredient to the blender. Blend for five minutes. Pass the magnesium stearate through a security screen and add this ingredient to the blender. Blend for three minutes. Fill the finished powders into hard gelatin capsules.

Formulation 2

| Ingredient | Mg per capsule |
|---|---|
| Crystalline Non-Solvated GS II Mesylate | 122.1 |
| Lactose monohydrate | 119.4 |
| MCC | 52.5 |
| Pre-gelatinized Starch | 35.0 |
| SLS | 7.0 |
| Sodium Starch Glycolate | 10.5 |
| Sodium stearyl fumarate | 3.5 |
| TOTAL | 350 |

Make a 10% by weight SLS aqueous spray solution. Screen the lactose monohydrate, the MCC, the crystalline non-solvated GS II mesylate and the pre-gelatinized starch through a security screen and transfer the ingredients into a granulator bowl. Add the spray solution to the blended powders while mixing. After complete addition of the spray-solution, switch to water and continue spraying until granulation process is complete. Spread the granulated materials on paper lined trays for drying or dry the granulated materials in a fluid bed dryer. Remove the dried granulation and mill. Place the milled material into a suitable blender. Pass the sodium starch glycolate through a security screen and add this ingredient to the blender. Blend for 3 minutes. Pass the sodium stearyl fumarate through a security screen and add this ingredient to the blender. Blend for 5 minutes. Fill the finished powders into hard gelatin capsules.

### Biological Assays

Ishikawa Cell Proliferation Assay:

This assay measures cell proliferation in both an agonist mode in the presence of a salt of the present invention alone, and in an antagonist mode in which the ability of a salt of the present invention to block estradiol stimulation of growth is measured. This assay utilizes Ishikawa human endometrial tumor cells and measures agonist and antagonist effects on endogenous ER receptors via an alkaline phosphatase endpoint (Littlefield et al., Endocrinology, 127:2757-2762, 1990). Alkaline phosphatase activity is measured as an endpoint for relative estrogenic stimuli in both an agonist mode and antagonist mode, where the ability of a test compound to block estradiol stimulatory activity is measured (Bramlett, K S, Burris, J., Steroid Biochem. Molec. Biol., 86:27-34, 2003).

Ishikawa cells are maintained in MEM (minimum essential medium, with Earle's salts and L-Glutamine, Gibco BRL, Gaithersburg, Md.), supplemented with 10% fetal bovine serum (FBS) vol/vol, (Gibco BRL). One day prior to assay, growth media is changed to assay medium, DMEM/F-12 (3:1) supplemented with 5% dextran coated charcoal stripped fetal bovine serum (DCC-FBS) (Hyclone, Logen, Utah), L-Glutamine (2 mM), MEM sodium pyruvate (1 mM), HEPES (N-[2-hydroxyethyl]piperizine-N′-[2-ethanesulfonic acid] 2 mM) all from Gibco BRL). After an overnight incubation, Ishikawa cells are rinsed with Dulbecco's Phosphate Buffered Saline (1×) (D-PBS) without Ca2+ and Mg2+ (Gibco BRL), and trypsinized by a 3-minute incubation with 0.25% Trypsin/EDTA, phenol red-free (Gibco BRL). Cells are resuspended in assay medium and adjusted to 250,000 cells/mL. Cells are added to flat-bottom 96 wells microculture plates at a density of 25,000 cells per 100 μL medium (Costar 3596) and incubated at 37° C. in a 5% CO2 humidified incubator for 24 hours.

The next day, serial dilutions of test compound are prepared in assay medium (at 6-fold the final concentration in the assay). For the agonist mode, plates received 25 μL/well of assay medium followed by 25 μL/well of diluted test compound (at 6-fold the final concentrations). For the antagonist mode, plates received 25 μL/well of 6 nM E2 (β-estradiol, Sigma, St. Louis, Mo.) followed by 25 μL/well of diluted test compound (at 6-fold the final concentrations). After an additional 48 hour incubation at 37° C., medium is aspirated from wells and 100 μL fresh assay medium added to each microculture. Serial dilutions of test compound is prepared and added to the cells as described above. After an additional 72 hour incubation at 37° C., the assay is stopped by removing medium and rinsing plates twice in Dulbecco's Phosphate Buffered Saline (D-PBS, Gibco BRL).

The plates are dried for 5 minutes and frozen at −70° C. for at least 1 hour. The plates are then removed from the freezer and allowed to thaw at room temperature. To each well, 100 μL of a 1:1 solution of 1-Step™ PNPP (Pierce Chemical Company, Rockford, Ill.) and DPBS (Gibco) is added. After a twenty minute incubation, plates are read on a spectrophotometer at 405 nm. The data are fitted to a linear interpolation to derive IC50 values for antagonist mode. For the agonist mode, a percentage efficacy for the test compound is calculated versus the response to 100 nM tamoxifen alkaline phosphatase stimulation as: 100×(test compound-control)/(tamoxifen-control). For the antagonist mode, a percentage efficacy for the test compound is calculated versus E2 (1 nM) alone as: 100×(E2-test compound)/(E2-control).

For the two assays that were run with crystalline non-solvated GS II mesylate, the antagonist response was 100% (±0.6%) with an IC50 of 2.4 nM (±0.4), and the agonist response was 13.8% (±11.5%).

3-Day Rat Uterine Antagonist

Female Sprague Dawley (SD) rats, 6 per group and 19 to 21 days of age, are orally treated with ethinyl estradiol (EE; 0.1 mg/kg) and 10, 1, 0.1, or 0.01 mg/kg test compound for 3 days. Test compound is dissolved in 20% w/v β-hydroxycyclodextrin in water and administered by oral gavage in a volume of 0.2 mL daily (15 minutes prior to the EE gavage). Groups of 6 rats are also given vehicle as a negative control and EE alone as a positive control. The animals are fasted overnight following the final dose. On the following morning, the animals are weighed and then euthanized (by carbon dioxide asphyxiation) and the uteri are rapidly collected (via a midline ventral incision), stripped of adipose tissue, removed luminal fluid by blotting onto absorbant paper, and weighed.

Uterine weight/body weight ratios (UWR) are calculated for each animal. The percentage inhibition of the estrogen-induced response is then calculated by the following formula: percent inhibition=100×(UWR_{EE}−UWR_{test compound}/UWR_{EE}−UWR_{control}). ED₅₀ values are derived from a semi-log regression analysis of the linear aspect of the dose response curve. For the two assays that were run with crystalline non-solvated GS II mesylate, the ED₅₀ is 0.22 mg/kg on both occasions.

8-Week Mature OVX Rat Uterine and Bone Effects

Virgin 6-month-old, SD female rats (Harlan, Ind.) weighing about 270 g are randomized to treatment groups and bilateral ovariectomies are performed using isoflurane anesthesia. Treatment is initiated 3 days after ovariectomy. Groups are orally dosed each day for 8 weeks with GS II mesylate in a vehicle of 20% hydroxypropyl-β-cyclodextrin (Aldrich Chemical Co., Milwaukee, Wis.). Vertebrae and femora are excised at necropsy and the mid-transverse section of the lumbar vertebra L-4 and distal femur metaphysis is scanned in 50% ethanol/saline, using quantitative computed tomography (QCT) (Research M, Norland/Stratec, Pt. Atkinson, Wis.). Cross-sectional area (X-Area), bone mineral content (BMC, mg), and volumetric BMD (vBMD, mg/cm³) are quantitated, using voxel dimensions of 148×148×500 μm as previously described (Sato M., Bone, 17:157S-162S, 1995). Bone measurements are carried out by computed tomography (CT) scans on the distal femur metaphysis (cancelous bone measurement) and the 5^{th} lumbar vertebrae (Sato, 1995 and Sato, et al., J. Med. Chem., 42:1-24, 1999).

Changes in uterine wet weight after 8 weeks of treatment with crystalline GS II mesylate were minimal. In comparison to ovary intact (sham operated) rats, dosing of ovariectomized rats with crystalline non-solvated GS II mesylate resulted in only 8.7% stimulation over OVX control at the 10-mg/kg dose. In addition, there was a significant preservation of both BMC and BMD even at a dose as low as 0.01 mg/kg of crystalline non-solvated GS II mesylate. BMD preservation was also seen in the vertebrae of the rats, although statistical significance for BMC changes were not achieved in the vertebrae.

### Utilities

The terms “treating” and “treat” as used herein, means alleviating, ameliorating, prohibiting, restraining, slowing, stopping, or reversing the progression or severity of a pathological condition, or sequela thereof, described herein. The term “preventing” refers to reducing the likelihood (risk) that the recipient of GS II mesylate, preferably crystalline non-solvated GS II mesylate, will incur, develop or re-incur (secondary prevention) any of the pathological conditions, or sequela thereof, described herein. A preferred mode of prevention in the context of endometriosis and/or uterine fibroids is secondary prevention.

The diseases, disorders or conditions for which GS II mesylate is useful in treating include, (1) uterine and/or breast cancer; (2) endometriosis; (3) uterine leiomyoma/leiomyomata; and (4) osteoporosis. Treatment of uterine leiomyoma/leiomyomata as described herein, may also reduce associated symptoms such as pain, urinary frequency, and uterine bleeding.

A “patient in need” or “woman in need” of the therapies described herein is a patient/woman either suffering from the claimed pathological condition, or sequela thereof, or is a patient/woman at a recognized risk thereof as determined by medical diagnosis, i.e., as determined by the attending physician.

### Dose and Route of Administration

As used herein, the term “effective amount” means an amount of a salt of the present invention that is capable of treating or preventing the conditions described herein.

The specific dose administered is determined by the particular circumstances surrounding each situation. These circumstances include: the route of administration, the prior medical history of the recipient, the pathological condition or symptom being treated or prevented, the severity of the condition/symptom being treated, and the age of the recipient. The recipient patient's physician should determine the therapeutic dose administered in light of the relevant circumstances.

When administered via the oral route, an effective minimum daily dose of crystalline non-solvated GS II mesylate will exceed about 15 mg. Typically, an effective maximum daily dose in this context (oral delivery) will not exceed about 240 mg. The exact dose may be determined, in accordance with the standard practice in the medical arts of “dose titrating” the recipient; that is, initially administering a low dose of the compound, and gradually increasing the dose until the desired therapeutic effect is observed.

Crystalline non-solvated GS II mesylate is preferably administered by the oral route.

### Combination Therapy

GS II mesylate, preferably crystalline non-solvated GS II mesylate, may be used in combination with other drugs that are used in the treatment of the diseases or conditions for which these compounds are useful (noted above). Such other drug(s) may be administered, by a route and in an amount commonly used therefore, contemporaneously or sequentially with GS II mesylate. When GS II mesylate is used contemporaneously with one or more other drugs, a pharmaceutical unit dosage form containing such other drugs in addition to the present compound is preferred. Accordingly, the pharmaceutical compositions of the present invention include those that contain one or more other active ingredients.

One example of another other active ingredient that may be combined with a compound of the present invention, either administered separately or in the same pharmaceutical composition, includes agents employed in the treatment of endometriosis and/or uterine leiomyoma such as leuprolide acetate, danazol, prescription and over-the-counter pain relievers and progestin-only oral contraceptives, or progesterone receptor modulators.

1. 1-(4-(2-Piperidinylethoxy)phenoxy)-2-(3-hydroxyphenyl)-6-hydroxynaphthalene methanesulfonic acid salt.

2. The salt of claim 1 that is crystalline and non-solvated.

3. The salt of claim 2 having an X-ray diffraction pattern which comprises the following peaks: 13.0±0.1, 13.6±0.1, 18.6±0.1, 19.0±0.1, 21.0±0.1 and 22.3±0.1° in 2θ; when the pattern is obtained from a copper radiation source (CuKα λ=1.54056 Å).

4. The salt of claim 3 having an X-ray diffraction patten which further comprises the following peaks: 6.4±0.1, 7.9±0.1 and 9.3±0.1° in 2θ.

5. A method of treating endometriosis comprising administering to a woman in need thereof an effective amount of a salt of claim 1.

6. A method of treating uterine leiomyoma comprising administering to a woman in need thereof an effective amount of a salt of claim 1.

7. (canceled)
